(19) United States
(12) Patent Application Publication
(10) Pub. No.: US 2004/0030402 A1
(43) Pub. Date: Feb. 12, 2004

(54) Elastomeric covers for orthopedic implants

(76) Inventors: Uri Arnin, Kiryat Tivon (IL); Yossi Tuval, Netanya (IL)

Correspondence Address: Dekel Patent Ltd., Beit HaRofim, Room 27, 18 Menuha VeNahala Street, Rehovot, IL

(21) Appl. No.: 10/633,501

(22) Filed: Aug. 5, 2003

Related U.S. Application Data
(60) Provisional application No. 60/401,530, filed on Aug. 8, 2002.
(60) Provisional application No. 60/411,933, filed on Sep. 20, 2002.
(60) Provisional application No. 60/426,345, filed on Nov. 15, 2002.

(51) Int. Cl. A61F 2/36

(52) U.S. Cl. 623/23.12

(57) ABSTRACT

An elastomeric cover for an orthopedic implant, the elastomeric cover having a seamless articulating surface which is at least part of a three-dimensional curved surface, wherein a cross-section of a portion of the seamless articulating surface subtends an arc greater than 180 degrees. Methods for forming the elastomeric cover are disclosed.

### CROSS-REFERENCE TO RELATED APPLICATIONS

This application claims priority under 35 USC. §119 to U.S. Provisional Patent Application Serial No. 60/401,530, filed on Aug. 8, 2002, U.S. Provisional Patent Application Serial No. 60/411933, filed Sept. 20, 2002, and U.S. Provisional Patent Application Serial No. 60/426345, filed Nov. 15, 2002, all of which are incorporated herein by reference.

### FIELD AND BACKGROUND OF THE INVENTION

The invention relates to elastomeric covers for orthopedic implants and methods of producing same and orthopedic implants including same. More particularly, the invention relates to implantable artificial joints and methods of making them, for example, cushioned ball components that may be used to replace the natural ball in a ball-and-socket joint, for example, in a hip or shoulder joint.

An artificial ball may be used, for example, to replace the femoral or humeral head of a patient in a partial or total hip or shoulder arthroplasty surgery. A soft-cushioned ball (or “head”) is generally formed of a metal ball covered by an elastomeric shell that replaces a naturally articulating surface of a joint such as the hip or shoulder. When an artificial head contacts cartilage in a joint, a cushioned, compliant outer layer on the ball may reduce damage caused by the endoprosthesis to the native cartilage, prolong the lifetime of the articular cartilage, and aid in maintaining the overall condition of the joint following implantation of the artificial joint component.

It may be difficult to create a successful soft-cushioned head due to the presence of parting lines on the outside surface of the covering that may irritate the native cartilage. In addition, in case of a total hip replacement, the parting lines on a soft-cushioned head may constantly rub against a rigid cup and could cause premature deterioration of the soft-cushioned head.

### SUMMARY

In one embodiment, an elastomeric cover is provided for an orthopedic implant, the elastomeric cover having a seamless articulating surface at least as great as a hemisphere.

In accordance with an embodiment, the elastomeric cover includes a mold-parting seam on a non-articulating surface thereof.

Further in accordance with an embodiment, the non-articulating surface includes an inner surface of the elastomeric cover that is adapted to contact an outer surface of an orthopedic implant.

In accordance with another embodiment, the elastomeric cover may include an extraneous portion extending away from the seamless articulating surface. The extraneous portion may be removable prior to implantation of the orthopedic implant in a recipient.

There is also provided in accordance with an embodiment an orthopedic implant at least partially covered with an elastomeric cover, the elastomeric cover having a seamless articulating surface at least as great as a hemisphere. The elastomeric cover may be produced by at least one of injection molding and blow molding.

### BRIEF DESCRIPTION OF THE DRAWINGS

With specific reference now to the drawings, it is stressed that the particulars shown in the drawings are by way of example and for purposes of illustrative discussion of the preferred embodiments of the present invention only, and are presented to provide what is believed to be the most useful and readily understood description of the principles and conceptual aspects of the invention. Thus, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a perspective view of an embodiment of an orthopedic implant covered by a pliant elastomeric cover;

FIG. 2 is a cross-sectional view of an elastomeric cover fabricated with a seam;

FIGS. 3-6 are cross sectional views of embodiments of optional molds; and production methods; and

FIG. 7 is a simplified flow diagram illustrating embodiments of production methods.

### DESCRIPTION OF THE PREFERRED EMBODIMENTS

Elastomeric covers for orthopedic implants, methods of producing such covers and methods of producing orthopedic implants with such covers are disclosed. The covers can be used to reduce contact stress between opposing articulated surfaces. Specifically, in implantable artificial joints, for example, cushioned ball components may be used to replace the natural ball in a ball-and-socket joint, such as a hip or shoulder joint. The principles and operation of elastomeric covers for orthopedic implants and methods of producing same and orthopedic implants including same according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As stated above, the technological feats in producing a seamless elastomeric cover for an orthopedic implant less than a hemisphere such as in a shoulder ball and socket joint can be overcome by simply producing an elastomeric cover by injection or blow molding with any seam in the portion of the sphere that is between the portion to be used in the implant and the total elastomeric cover that is produced.

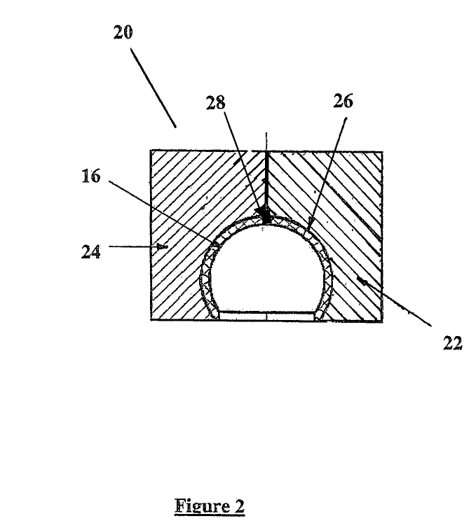

However, it has previously been considered impractical to produce a mold without creating a seam on an articulating surface that is greater than a hemisphere (180°). Referring to FIG. 2, in order to produce a partial spherical covering 16 of greater than 180 degrees, normally a mold 20 is used that has at least two pieces 22 and 24. Thus, the material for forming the covering may be injected or blown into the mold cavity 26 to form the shape of cover 16 and then cavity 26 may be opened to remove covering 16 from mold 20. However, the mating surface between parts 22 and 24 of mold 20 creates a seam 28 on covering 16 known as a parting line 28. A parting line on the outside surface 17 of covering 16 is unacceptable in the case of using the covering for orthopedic articulating applications because any surface blemish can lead to the abovementioned contact stresses that can cause joint wear and patient discomfort when used clinically in the case of a partial hip arthroplasty or a premature wearing away of the elastomeric cover when the opposing articulating socket surface is an artificial rigid socket component in the case of a total hip replacement.

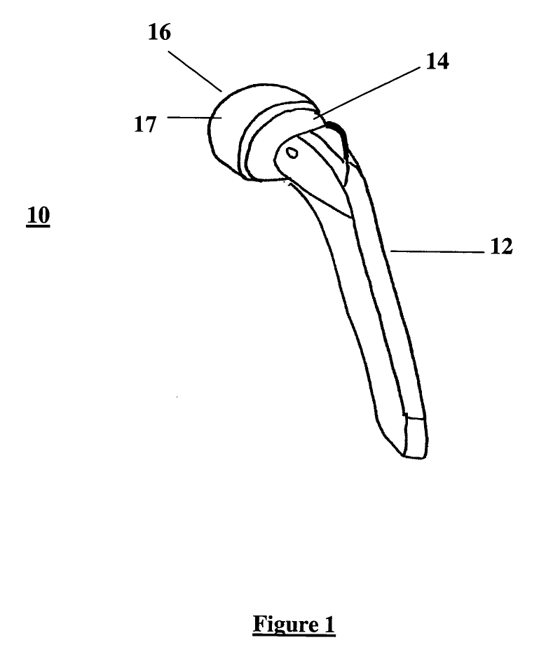

Reference is now made to FIG. 1, which illustrates a typical orthopedic implant 10. A ball and stem configuration that may be used for example in a hip joint is shown. In the hip joint, the stem 12 is inserted into the central core of the femur. The ball component 14 of the ball and stem configuration of the part is the component that articulates. The surface of ball component 14 is covered by a compliant (elastomeric) material 16. The outer surface 17 of the complaint elastomeric material 16 is the articulating surface. It is noted that an “articulating surface” refers to the contact surfaces of a joint, wherein at least one of the contact surfaces moves with respect to the other contact surface to form a movable joint. A “non-articulating surface” refers to a portion that does not contact such contact surfaces of the joint.

The attachment of a cushioning, elastomeric cover 16 to a rigid sub-layer or sub-layers enhances the performance of a prosthesis. The cover 16 may be made from, for example, polyurethane containing materials, silicone containing materials, polyethylene based elastomers, hydrogels, and polypropylene containing materials, and the underlying ball component 14 may be made of metal, including, stainless steel, titanium alloy, cobalt chromium alloys, ceramics, or other hard, rigid materials.

The ball component 14 may have different shapes and sizes depending on the application. For example, ball component 14 may have a partial-sphere shape having a radius that depends on where the ball is used. The radius may be larger when used in a large patient. The shape of the ball may be about 200-265 degrees of a full sphere, when used in the hip, for example. The ball component 14 may be connected to a stem 12 using any one of numerous methods known to those skilled in the art.

The soft cushioning cover layer 16 is generally made of an elastomeric material. By “elastomeric” it is meant an elastic material able to generally resume its original shape when a deforming force is removed, such as but not limited to, natural rubbers, synthetic rubbers, polycarbonate urethane copolymers, silicone-containing materials, polyethylene-based materials, hydrogels, and polypropylene-containing materials. The layer is of a hollow, partial sphere shape, comprising about the same fraction of a full sphere as the hard, metal ball does.

Accordingly, the elastomeric cover 16 has a seamless articulating surface, which is at least part of a three-dimensional curved surface (such as but not limited to, a sphere, ellipsoid, paraboloid and the like), wherein a cross-section of a portion of the seamless articulating surface subtends an arc greater than 180 degrees (such as but not limited to, a hemisphere.) By “seamless” it is meant that the surface is substantially devoid of a seam from a molding process.

The layer 16 may have different shapes and thicknesses. For example, the layer 16 may have a hollow spherical shape extending past its equator (past 180 degrees) with a thickness of about 1 mm, and more particularly more than about 1.5 mm, more than about 2 mm, more than about 2.5 mm, more than about 3 mm, more than about 3.5 mm. The compliant layer may also have a thickness of generally about 4 mm, and more particularly less than about 3.5 mm, less than about 3 mm, less than about 2.5 mm, less than about 2 mm, and less than about 1.5 mm. The material hardness of the compliant material is between about 60 Shore A to about 65 Shore A. More particularly, the material hardness may be greater than about 60 Shore A, greater than about 65 Shore A, greater than about 70 Shore A, greater than about 75 Shore A, greater than about 80 Shore A, greater than about 85 Shore A, greater than about 90 Shore A, or greater than about 95 Shore A. The material hardness may also be less than about 105 Shore A, less than about 95 Shore A, less than about 90 Shore A, less than about 85 Shore, less than about 80 Short A, less than about 75 Shore A, less than about 70 Shore A, or less than about 65 Shore A. The compliant material covering the hard ball has an elastic modulus of between about 10 to about 150 MPa. More particularly, the material may have an elastic modulus of greater than about 10 MPa, greater than about 30 MPa, greater than about 50 MPa, greater than about 70 MPa, greater than about 90 MPa, greater than about 110 MPa, greater than about 130 MPa, and may have an elastic modulus of less than about 150 MPa, less than about 130 MPa, less than about 110 MPa, less than about 90 MPa, less than about 70 MPa, less than about 50 MPa, or less than about 30 MPa. The outside surface of the elastomrnc covering 16 may have a smooth surface or it may have a non-smooth surface, such as some modifications to a generally smooth surface to improve its lubrication quality when in use, e.g., grooves or a slightly rough texture. Scoring the surface slightly with shallow grooves may facilitate lubrication by providing passageways for retaining synovial fluid on the surface.

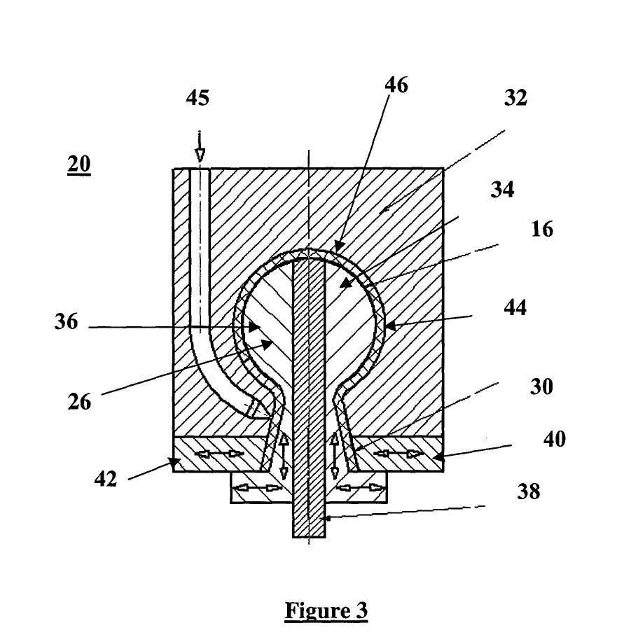

Referring to FIG. 3, to produce a partially spherical covering 16 of greater than 180 degrees without a parting line 28, the hollow partial-sphere may include a temporary neck 30 when it is formed in the mold 20. Neck 30 can be of different shapes, full or partial, and may allow material to flow into the mold 20 in the case of injection molding and allow the part to be pulled out after it has been formed.

The mold 20 may be formed of six parts (e.g. 32; 34; 36; 38; 40 and 42) as shown in FIG. 3 such that no parting lines 28 are formed on the articulating surface 44 of elastomeric, partially spherical covering 16. The mold 20 may be assembled by inserting 4 to 8 mandrels 34 and 36 (two are shown) into the cavity of part 32 and then inserting a core 38 between mandrels 34 and 36 to separate mandrels 34 and 36. Parts 40 and 42 may be then positioned to create a closed cavity in the shape of a hollow partial-sphere with neck 30. An injection molding process may be then used to create the hollow partial sphere without a parting line 28. Material may be injected through the gate 45 into the cavity at an injection pressure of about 300-1700 bar at a temperature of about 180-230 Celsius degrees, while the mold has a temperature of about 30-90 Celsius degrees. The injection pressure may be maintained for about 1-20 seconds and the holding pressure may be maintained for about 5-50 seconds. The hot material is then allowed to cool for about 15-100 seconds. After the clamping pressure is released, the mold 20 may be disassembled by removing the core part 38 and parts 40 and 42. Mandrels 34 and 36 may be slipped out of the mold cavity, after which the formed elastomeric cover 16 (still including neck 30) may be removed from the mold cavity by grasping neck 30 and withdrawing the partial sphere without touching the hollow sphere, the useful portion 46.

After the covering has been removed from the mold 20, neck 30 may be cut to remove the extraneous portion of cover 16, so that partially hollow sphere 46 may be ready for use. Because part 32 of mold 20 may be a single piece construction, a parting line 28 does not exist on the finished product. Parting lines 28 may exist on neck 30 where mold parts 32; 34; 36; 38; 40; 42 and 44 contact each other and where material may be injected during formation of part 16, but no parting lines 28 exist on the articulating surface 44 of hollow partial sphere 46.

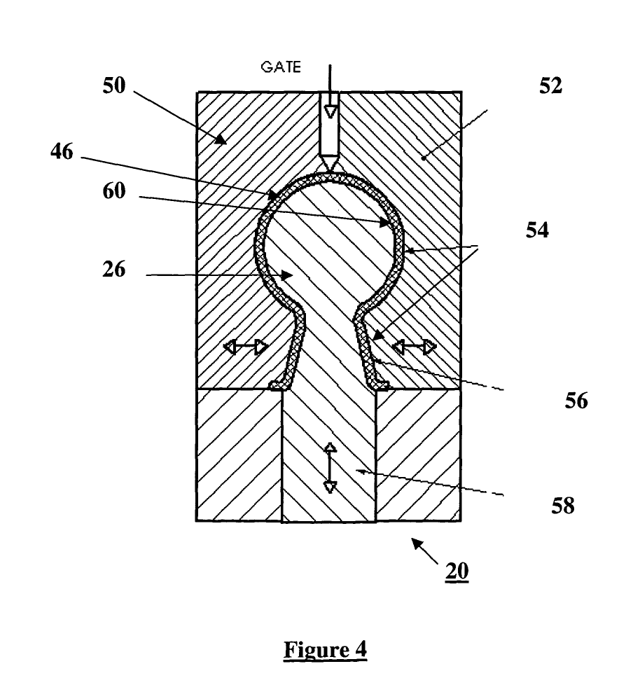

Referring to FIG. 4, the compliant covering with a seamless articulating surface may be formed through an “inside-out” injection molding process using a multi-part mold 20. The cavity 26 may be formed of multiple mold parts (e.g., 50 and 52) and the useful part 54 of covering 46 and the extraneous portion (the neck) 56 may be molded on a polished core 58 around which the multi-part mold fits. After cooling the part, the parts of the mold 20 may be separated, and the covering may be removed from the polished core by peeling it off and inverting it so that the surface 60 that was in contact with the polished core 58 during the molding process becomes the outside surface of the cover 46. Because polished core 58 may be a single piece, it does not create a parting line 28 or blemish on the covering. The neck 56 may then be cut away from the finished product as described hereinabove.

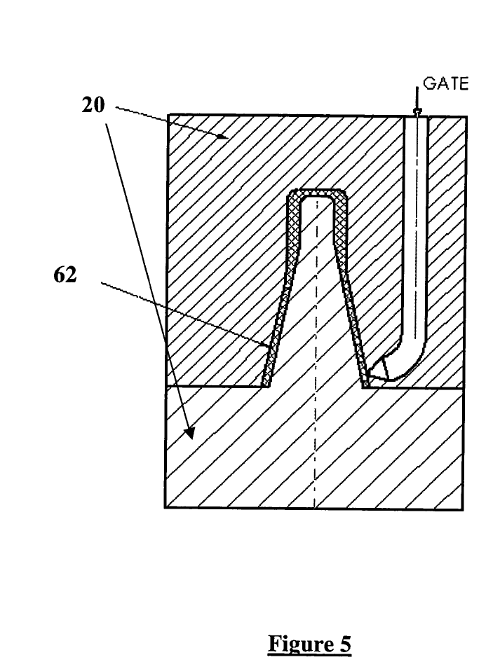

Referring to FIG. 5, a two-step blow molding process may be used to form the partial sphere without a parting line on the articulating surface. A pre-form of elastomeric material 62 may be made in a two-part mold 20 with no parting lines on the useful portion, using normal injection molding techniques.

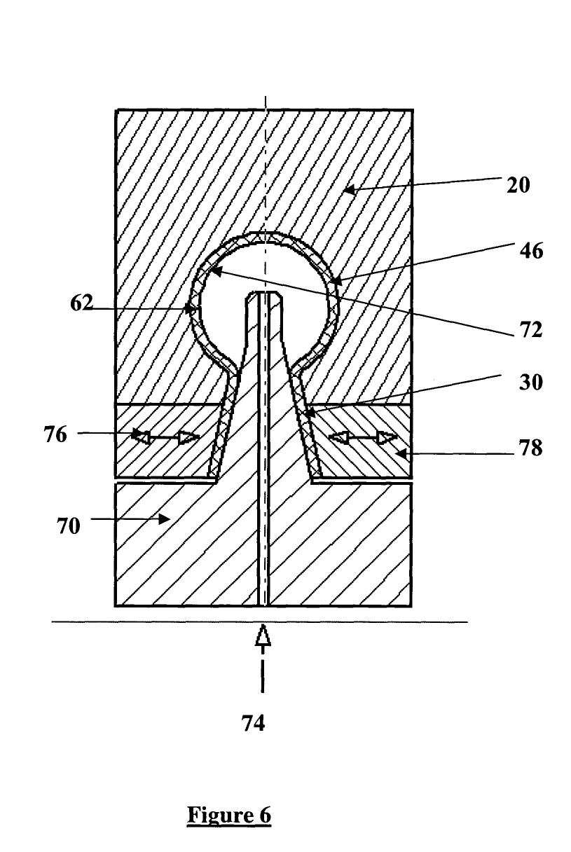

Referring to FIG. 6, the pre-form 62 may be used to mold a compliant partial-sphere covering without a parting line. The pre-form 62 may be placed in a cavity 72 of a second mold 20 and a mandrel 70 may be inserted within the pre-form 62. Pre-form 62 may be heated so that it becomes pliant and then compressed air may be blown into cavity 72 of second mold 20 through an orifice 74 in mandrel 70 to inflate pre-form 62 into a shape governed by the internal dimensions of cavity 72 of second mold 20. After cooling the part, mandrel 70 and other parts (e.g. 76 and 78) of the mold 20 may be removed and then the finished partially spherical part may be removed from the mold 20 cavity by grasping neck 30 and removing part 16 without touching the articulating surface 72 of sphere 54. Parting lines may exist on neck 30 where the mold parts contact each other, but no parting lines exist on the articulating surface of hollow partial sphere, the useful portion 46.

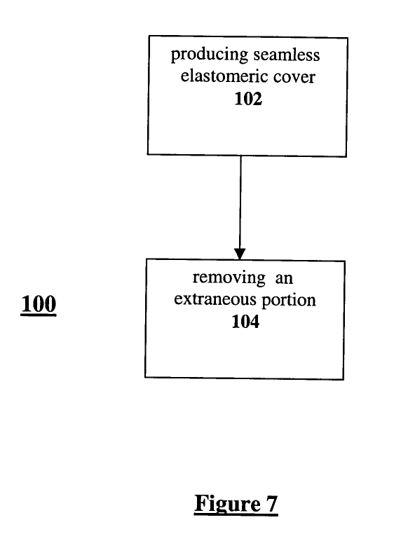

FIG. 7 illustrates a method 100 of producing an elastomeric cover 16 (such as the one shown in FIG. 1) for an orthopedic implant 14. Cover 16 reduces contact stresses between implant 14 and an opposing articulating socket surface in which orthopedic implant 14 may be implanted.

The method 100 may be as described hereinabove with reference to FIGS. 3-6. Accordingly, the method 100 may include producing 102 a seamless elastomeric cover designed and constructed to engage and retain an orthopedic implant in a form of at least approximately a hemisphere, and to conform to a surface of the orthopedic implant 14 such that cover 16 may be capable of reducing contact stresses between orthopedic implant 14 and an opposing articulating socket surface in which orthopedic implant 14 may be implanted. Method 100 further includes removing 104 an extraneous portion 30 from the useful portion 46 prior to the implantation of orthopedic implant 14 into an orthopedic implant recipient.

According to various preferred embodiments of the invention, producing 102 may be performed using any known molding method such as, for example, injection molding or blow molding (FIGS. 3-6).

In some cases opposing articulating socket surface may be a tissue of the recipient such as acetabular cartilage (hip socket) or glenoidal tissue (shoulder socket). In other cases the opposing articulating socket surface may be an artificial rigid socket component. The nature of opposing surface does not influence the function of cover 16 in any significant way.

A seam 28 resulting from producing 102 by the molding process may be present on, for example, extraneous portion 30 or on an inner surface of elastomeric cover, 16 for example on the inner surface of useful portion 46.

According to most preferred embodiments of the invention, extraneous portion 30 may be designed and constructed to facilitate extracting elastomeric cover 16 from a mold and inverting elastomer cover 16.

The present invention may be further embodied by an orthopedic implant 14 at least partially covered by elastomeric cover 16 as described hereinabove.

It may be appreciated that certain features of the invention, which may be, for clarity, described in the context of separate embodiments, may also be provided in combination in a single embodiment. Conversely, various features of the invention, which may be, for brevity, described in the context of a single embodiment, may also be provided separately or in any suitable sub combination.

Although the invention has been described in conjunction with specific embodiments thereof, it may be evident that many alternatives, modifications and variations will be apparent to those skilled in the art. Accordingly, it may be intended to embrace all such alternatives, modifications and variations that fall within the spirit and broad scope of the appended claims. All publications, patents and patent applications mentioned in this specification may be herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference may be available as prior art to the present invention.

What is claimed is:

1. An elastomeric cover for an orthopedic implant, said elastomeric cover having a seamless articulating surface which is at least part of a three-dimensional curved surface, wherein a cross-section of a portion of said seamless articulating surface subtends an arc greater than 180 degrees.

2. The elastomeric cover according to claim 1, wherein said seamless articulating surface comprises a portion of a sphere greater than a hemisphere.

3. The elastomeric cover according to claim 1, wherein said elastomeric cover comprises a mold-parting seam on a non-articulating surface thereof.

4. The elastomeric cover according to claim 3, wherein said non-articulating surface comprises an inner surface of said elastomeric cover that is adapted to contact an outer surface of rigid part of an orthopedic implant.

5. The elastomeric cover according to claim 3, wherein said non-articulating surface comprises an extraneous portion extending away from said seamless articulating surface.

6. The elastomeric cover of claim 5, wherein said extraneous portion is removable prior to implantation of said orthopedic implant in a recipient.

7. An orthopedic implant at least partially covered with an elastomeric cover, said elastomeric cover having a seamless articulating surface which is at least part of a three-dimensional curved surface, wherein a cross-section of a portion of said seamless articulating surface subtends an arc greater than 180 degrees.

8. The orthopedic implant according to claim 7, wherein said seamless articulating surface comprises a portion of a sphere greater than a hemisphere.

9. The orthopedic implant according to claim 7, wherein said elastomeric cover comprises a mold-parting seam on a non-articulating surface thereof.

10. The orthopedic implant according to claim 7, wherein said elastomeric cover is produced by at least one of injection molding and blow molding.

11. The orthopedic implant according to claim 7, wherein said elastomeric cover has a thickness in a range of about 1 mm to about 4 mm.

12. The orthopedic implant according to claim 7, wherein said elastomeric cover has a material hardness in a range of about 60 Shore A to about 95 Shore A.

13. The orthopedic implant according to claim 7, wherein said elastomeric cover has an elastic modulus in a range of about 10 to about 150 MPa.

14. The orthopedic implant according to claim 7, wherein said elastomeric cover has a smooth surface.

15. The orthopedic implant according to claim 7, wherein said elastomeric cover has a non-smooth surface.

16. A method forming an elastomeric cover for an orthopedic implant, the method comprising:
forming an elastomeric cover having a seamless articulating surface which is at least part of a three-dimensional curved surface, wherein a cross-section of a portion of said seamless articulating surface subtends an arc greater than 180 degrees.

17. The method according to claim 16, further comprising:
forming a mold having a closed cavity in the shape of said seamless articulating surface;
injecting material into said cavity to form said elastomeric cover having a seamless articulating surface, said material also forming an extraneous portion extending away from said seamless articulating surface; and
removing said elastomeric cover from the mold.

18. The method according to claim 16, comprising forming said seamless articulating surface as a portion of a sphere greater than a hemisphere.

19. The method according to claim 17, further comprising forming a mold-parting seam on a non-articulating surface of said elastomeric cover.

20. The method according to claim 17, further comprising detaching said extraneous portion prior to implantation of said orthopedic implant in a recipient.

21. The method according to claim 16, wherein said elastomeric cover is formed by at least one of injection molding and blow molding.
